(19) United States
(12) Patent Application Publication
(10) Pub. No.: US 2008/0086785 A1
(43) Pub. Date: Apr. 10, 2008

(54) Isolated nucleic acid sequence conferring stress tolerance/susceptibility in rice

(75) Inventors: Villoo Morawala Patell, Bangalore (IN); Mathai Chettoor Antony, Bangalore (IN); Divya Chandran, Bangalore (IN); Ashok Madurappa, Bangalore (IN)

Correspondence Address: SIDLEY AUSTIN LLP;ATTN: DC PATENT DOCKETING, 1501 K STREET, N.W., WASHINGTON, DC, 20005, US

(73) Assignee: Avestha Gengrain Technologies Pvt. Ltd.

(21) Appl. No.: 11/657,580

(22) Filed: Jan. 25, 2007

Related U.S. Application Data
(63) Continuation of application No. 09/868,025, filed on Feb. 19, 2002, now abandoned.

(30) Foreign Application Priority Data
Oct. 13, 1999 (IN) 997/MAS/99

(51) Int. Cl. A01H 5/00; C07H 21/04; C12N 15/82

(52) U.S. Cl. 800/278; 536/23.1; 800/298

(57) ABSTRACT

The present invention relates to an isolated nucleic acid sequence AGT-SAL 11 encoding polypeptides which confers salt tolerance on plants and other organisms.

### CROSS-REFERENCE TO RELATED APPLICATIONS

The present application is a continuation application of U.S. application Ser. No. 09/868,025, filed Feb. 19, 2002, currently pending, which is incorporated herein by reference in its entirety.

### BACKGROUND OF THE INVENTION

1. Field of the Invention

The present invention relates to an isolated nucleic acid sequence conferring stress tolerance/susceptibility in rice. More specifically this invention relates to a method for conferring salt tolerance in plants. However, this gene can also be used to engineer stress tolerance in other plant species.

Altered gene expression lies at the heart of regulatory mechanisms that control cell biology. Comparisons of gene expression in different cell types provide the underlying information that analyzes the biological processes that control our lives. Effective methods are needed to identify and isolate those genes that are differentially expressed in various cells or under altered conditions.

2. Description of the Related Art

Life cannot exist without water. It forms an important constituent of the plant and animal cell and is present to the extent of 80 to 90%. Water is essential for plants for the following reasons:
1. It is the major component of protoplasm. If the protoplasm is dehydrated, it ceases to be active and the protoplasm loses its physical and chemical properties. Water maintains turgidity of cells.
2. Water is a universal solvent. The intake of minerals and nutrients from the external medium into the cell is only in the dissolved form.
3. Water serves as the medium for translocation of minerals from the soil to leaves through the xylem and food manufactured by the leaves to other plant parts via phloem.
4. Water also plays an important role in the transport of plant hormones.
5. Plant movements (especially of certain organs) are caused by changes in water content of cells.
6. Water is directly involved in the bio-chemical reactions that take place in plant cells. Hydrolysis of macromolecules takes place by the addition of water. Water is the source of hydrogen for the reduction of carbon dioxide during photosynthesis. Water is one of the products of cellular respiration. All these reactions are influenced by the availability of adequate and good quality water.

Since water plays such an important role in plants, its deficit severely affects cellular functions, plant growth and development and reduces yields. However, the plant devises a number of changes that occur at the whole plant, physiological, cellular, bio-chemical and molecular levels in an attempt to cope with moisture stress.

Furthermore, due to the widespread use of irrigation and limited water supply, many cultivated areas have become increasingly salinized. Irrigation imparts increased salt concentration when the available irrigation water evaporates and leaves previously dissolved salts behind.

Dissolved salts in the soil increase the osmotic pressure of the solution in the soil and tends to decrease the rate at which water from the soil will enter the roots. If the solution in the soil becomes too saturated with dissolved salts, the water may actually be withdrawn from plant roots. Thus the plants slowly starve though the supply of salts and dissolved nutrients may be more than ample.

Salinity and water deficit have shown to induce the expression of number of genes. These gene products have either regulatory role in gene expression or a functional role in adaptive responses of plant cells to the stress.

Salinity refers to the presence of various salts in soil and irrigation water in concentrations that affect the growth and yield of plants. Sodium chloride (common salt), is often the dominant salt present in saline soils. Saline-alkaline and sodic soils may have excess of chlorides, sulphates and bicarbonates of sodium, calcium and potassium in addition to other inorganic ions.

Saline soils have a soil water conductivity of 4 deci-seimen/meter and exchangeable sodium percentage of not less that 15. This translates into nearly 2.56 g/L of total dissolved salts in an extract or if all the salt is NACl, an ionic concentration of 44.14 mM.

The irrigation water in majority of the rice growing areas is generally of marginal or poor quality (EC of 2-5ds/m or more). Though water is present it is unavailable to plants because the osmotic potential of soil is altered. To exclude salts and minimize ion toxicity, water must be imported against a free energy gradient. However, if water is taken up freely, the endogenous salt concentration rises.

Macromolecular assemble and enzyme activity associated with shaping and maintaining each cell can proceed only with a properly constituted ionic environment. The inorganic ions selectively neutralize charges on macromolecular surfaces and simultaneously permit formation of intramolecular bridges that determine the final conformation of many proteins. The same ions also determine the availability of free water around enzymes and their substrates and thus the rate of catalysis. Finally, ionic gradients, set up at considerable cost to the plant cell, constitute free energy gradients that can be tapped to direct the flow of organic molecules and between cells [Claes et al., 90].

An extracellular ion excess invariably disrupts the ionic balance intracellularly. With the influx of salt, proteins may denature or aggregate leading to a loss of function, gradient-driven pumps may reverse and thus block the normal redistribution of symported molecules, membrane fluidity and consequently, the activity of some membrane components may change, and even the entry of water may be restricted. Some ions may have additional secondary effects. For example, increasing amounts of intercellular Na+can lead to decreases in the concentration of K+ [Ben-Hayyim et al., 1987; Binzel et al., 1988]. This, in turn, reduces the rate of photosynthesis [Pier and Berkowitz, 1987], and, based on studies with bacteria can accelerate polysome decay and degradation of the free ribosomal proteins [St. John and Goldberg, 1980]. Salt imposed stress has been shown to have an impact even before ions enter the cell. Extracellular Na+ (or mannitol), for example, can leach Ca²⁺ from root cell plasmalemma, and as a result of membrane destabilization, increases K+ efflux[Cramer et al., 1985].

These are only the immediate problems facing the cell. If the stress is prolonged, normal maintenance processes are impaired because general protein synthesis [Hurkman and Tanaka, 1987] and metabolism [Criddle et al., 1989] both decline. Denatured proteins may form inactive complexes with otherwise functional proteins. Enzymes may be poisoned when inorganic cofactors are displaced by incoming salts.

Rice is a salt sensitive plant and the most important cereal crop of the world. This crop is grown in diverse ecosystems and extensively in the tropics. Rice is the staple food of majority of the people in South & East Asian nations, parts of South America and Africa. The present production of rice in the world falls well short of the demand. To meet the ever-increasing demand, continuous improvement in the quality and productivity of this cereal is vital.

Several biotic and abiotic factors are important constraints in increasing the quality and yield of rice. Biotic stresses in the form of pests and diseases considerably affect rice productivity. Abiotic stresses however have been shown to cause more harm to the rice crop than biotic stresses. The major abiotic stresses, which significantly hamper rice yields are drought, salinity, floods, extremes of temperature and metal toxicity.

Minimizing crop losses by abiotic stresses especially drought and salinity is an important area for the overall improvement of rice. A thorough understanding of the responses of the rice plant to abiotic stresses is fundamental for developing a strategy to make the rice plant more hardy.

Initial work in understanding the effects of abiotic stress on rice was done at the whole plant level. The role, interactions and alterations of root-shoot characteristics in response to stress in rice has been studied. Later work focused on the physiology of stress. The effects of drought and salinity on the physiological processes like metabolism, growth and development has come forth.

Efforts have also been made to improve the performance of rice crop under limiting environmental conditions through traditional breeding programs and agronomic practices. Strategies for the evaluation of rice for drought and salinity tolerance using field screening and multi-location testing have been developed. These approaches have also been able to distinguish rice varieties into susceptible and tolerant ones. The development of molecular linkage maps and the use of molecular markers, of late, is helping selection and breeding for drought resistance. Molecular markers linked to root traits, osmotic adjustment and other stress tolerant characters are now being identified and used for selection and breeding.

The molecular responses of plants to abiotic stresses is a complex phenomenon. However, advances in molecular biology offer new tools to investigate changes in plants, at the cellular and molecular level, in response to abiotic stresses.

Relatively speaking, this species is more sensitive to salt stress at the seedling stage and the reproductive stage [Lutts et al., 1995]. Excess salt leads to reduced seed germination and poor seedling vigor. During the vegetative phase, premature senescence of leaves and reduced number of tillers can occur. During the reproductive stage, the number of spikelets per panicle get significantly reduced [Lutts et al., 1996].

Furthermore, rice cultivation in tropical areas is mostly dependent on seasonal rainfall, vagaries of tropical monsoon renders the growth and yield of rice crop uncertain. The modern high yielding varieties of rice in particular are unable to attain their full genetic potential in the absence of adequate and good quality water.

Drought occurs when there is insufficient soil water to be taken up by the plants over a period of time to meet its transpirational requirements. Sustained drought results in complete loss of free water and will result in desiccation and dehydration. Concentration of solutes in the cell leads to drop in cellular water potential. Loss of turgor leads to changes in the cell volume and membrane area. The crucial cell wall plasma membrane continuum is lost. An osmotic shock can cause extensive cell damage through disruption of membrane integrity and leakage of cellular contents. Cellular water deficit causes extensive damage to functional proteins and increases formation of misformed proteins. Impairment in the normal metabolic pathways leads to formation of toxic and highly reactive by products such as the reactive oxygen species. Many other cellular changes similar to those occurring during salt stress are also observed during drought.

In Rice, at the plant level, drought affects several developmental processes. Seed germination is non-uniform. At the Vegetative stage, canopy photosynthetic rates decrease drastically. Root growth is affected. Leaf rolling and leaf scorching is observed. At the reproductive stage, drought causes pollen sterility, small, thin and deformed anthers. Drought during anthesis causes inhibition of another dehiscence and pollen germination, reduced pollen viability, failure of the panicle to exert the flag leaf, resulting in loss of grain set. Water constraint during ripening causes incomplete grain filling [O'Toole and Moya, 1981].

Molecular Responses of Rice to Salinity and Moisture Stress.

Osmotic stress (such as salinity and Drought) leading to water deficit elicit complex molecular responses in plants. The events described here are common to all plants and also apply to Rice.

The molecular responses of plants to water deficit is dependent upon the type of stress (salinity/drought), severity of stress (mild/moderate or severe) and duration of stress (sporadic or chronic). A gradual onset of stress allows cellular mechanisms to adopt better while a sudden severe stress results in cellular damage and activates repair mechanisms. Plant factors such as genotype/variety, developmental stage (seed/seedling/vegetative or reproductive stage) and organ (root/shoot etc.) exposed to stress also influences the nature of response [Bray, 1997].

Molecular events during water deficit has been investigated using four major approaches [reviewed in Ingram and Bartels, 1996]:
1. Examining tolerant systems such as seeds and resurrection plants.
2. Analyzing mutants from genetic model species.
3. Analyzing the effects on agriculturally relevant plants.
4. By the targeted expression of drought related genes in vivo using transgenic plants.

The responses of plants to water deficit at the molecular level normally occur in the following sequence [Bray, 1993]:
1. Cellular perception of the stress.
2. Signal transduction events.
3. Alterations in the gene expression.
4. The role of gene products induced by salinity and drought in stress avoidance of tolerance.
   The Role of Gene Products Induced by Salinity and Drought.

Salinity and Water deficit have shown to induce the expression of a number genes. These gene products have either a regulatory role in gene expression or a functional role in the adaptive responses of plant cells to the stress.

Many genes have been identified and characterized to have a definite role in the response of plants to salinity and drought, and are induced by a complex mechanism of stress perception and signal transduction events. Stress related gene products have a role in moisture stress tolerance such as signaling molecules, regulatory proteins, protection of cellular structures, synthesis of osmoprotectants, ion sequestration, chaperon activity and protein stabilization, protein degradation, scavenging of accumulated toxins (especially reactive oxygen species), promotion of damage repair mechanisms, anti-pathogen activity and others.

Changes, in the tissue specific gene expression, are fundamental to the responses that occur during salinity and drought and influence many of the short and long term cellular changes that determine stress resistance or susceptibility. Northern Blot analysis, using stress related cDNA probes, offers a simple but powerful tool to monitor alterations in gene expression in roots and shoots, in response to salinity and water deficit, while comparing a susceptible and tolerant variety.

Furthermore, subtractive hybridization technique has been used for identifying and cloning differentially expresses mRNAs. The basic principle of subtractive hybridization involves the hybridization of cDNAs from one population in which mRNAs are differentially expressed to excess constitutively expressed cDNAs from another population. The sequence that are common to both the populations are removed using hydroxypatite chromatography, avidin-biotin binding or oligo-dT beads. Despite the enormous success of subtractive techniques in cloning different genes, this requires multiple subtraction steps. Therefore, a new strategy was developed which permits exponential amplification of cDNAs that differ in abundance in 2 populations is suppressed.

Differential display is also a power tool for analyzing gene expression, allowing genes to be isolated solely on changes in phenotype and without prior knowledge of protein or nucleic acid sequence. This technique is flexible and is a comprehensive method for detecting almost all genes expressed in a particular cell and for identification of differences in gene expression between different cell types in both mammal and plant systems. There is a simultaneous display of up and down regulated genes, it permits side-by-side comparisons of mRNA from different sources; only a few (g of RNA is required, compared to 50× or more for subtractive hybridization; highly reproducible and finally high speed of analysis.

This method involves the reverse transcription of the mRNA with oligo-dT primers anchored to the beginning of the poly (A) tail, followed by the polymerase chain reaction on the presence of a second 10mer, arbitrary in sequence. PCR primers and conditions are chosen such that any given reaction yields a limited number of amplified cDNA fragments permitting their visualization as discrete bands following Gel Electrophoresis. The amplified cDNA sub-populations of 3′ termini of mRNAs as defined by this pair of primers are distributed on a DNA sequencing gel and visualized by autoradiography. Each pair of the primer produces a distinct pattern of bands. The band pattern obtained with each primer is compared. Differentially expressed bands are cut out of the gel and the DNA is eluted and re-amplified. The amplified products are cloned into suitable vectors and their sequence deduced.

### BRIEF SUMMARY OF THE INVENTION

The object of the present invention is to correlate the expression pattern (at the mRNA levels) of genes under study with their role in abiotic stress tolerance or susceptibility in IR64 (susceptible variety) and RASI (tolerant variety).

Yet another object of the present invention is to compare the differences in the expression of genes encoding stress proteins during salinity and desiccation.

Further object of this invention is to assess the gene expression pattern in root and shoot during different stages of salt and dehydration.

To achieve the said objects, the present invention relates to relates to a nucleic acid sequence comprising a polynucleotide, AGT-SAL 11 having a sequence SEQ ID NO: 1.

The AGT-SAL11 polynucleotide sequence encodes a polypeptide as shown in SEQ ID NO:2.

The polynucleotide has glycosylation and phosphorylation sites. The said glycosylation is 0 glycosylation.

Said AGT-SAL 11 has a mixture of αβ type of secondary structure.

The present invention further relates to a method for conferring salt tolerance on a plant, the method comprising introducing into the plant a recombinant expression cassette comprising a plant operator operably linked to AGT-SAL 11 polynucleotide sequence.

### BRIEF DESCRIPTION OF THE DRAWINGS

FIG. 1 is a photograph of an RNA agarose (denaturing gel) showing two prominent RNA bands of size 4.7 kb and 1.9 kb corresponding to 285 and 185 ribosomal RNA activity.

FIG. 2 is a photograph of sample RNA agarose (denaturing gel) with size markers.

### DETAILED DESCRIPTION OF THE INVENTION

Two Indian varieties of rice IR64 and RASI were taken. While IR-64 is susceptible to high salt stress, RASI is resistant to the same. The differential display technique was used to determine the regulation of gene expression at the cellular level in these two varieties under salt stress conditions and isolate the genes responsible for susceptibility or resistance in IR-64 and RASI respectively.

IR-64 and RASI seeds were subjected to salt stress using 150 mM NaCl. The RNA was isolated from both stressed plants and unstressed controls. Further processing of the RNA was done following the protocol provided by Gen-Hunter's differential display kit. The RNA was reverse transcribed using H-T11 primers to obtain the cDNA. This DNA was amplified by PCR using H-T11 primers and an arbitrary primer H-API. The PCR products were resolved on a 6% denaturing polyacrylamide gel and subjected to autoradiography. The autoradiogram showed 54 differentially expressed bands. The band labeled A-11 was cut out from the gel and DNA eluted. Reamplification of the DNA was done using the same primer set and PCR conditions.

The PCR product of AGT-SAL was cloned into TOP TA cloning vector, which is a unique, fast and an efficient way to clone PCR products. The vectors are linearized having an extra 3'T overhang and are activated with topoisomerase. Ligation takes advantage of the template independent addition of a single adenosine (A) to the 3′ end of the PCR products by Taq DNA Polymerase. The positive clones were checked for the presence of insert by digesting with EcoRI restriction endonuclease.

Two clones showing insert release were subjected to sequencing using sequences of the vector that flank the insert sites as primers; M13 forward and reverse primers shows that the sequence of interest lies between nucleotides 130 and 310 which ends with a stretch of poly A's. Since the fragment of interest was amplified using specific oligo-dT primers, its position in the sequence was located by searching for a poly A stretch downstream. This stretch was found around position 310, indicating the 3′ end of the sequence of interest.

For expressing the vector, the gene AGTSAL-11 (Accession No. AF 192975) should be cloned in expression vector where the protein of interest would be induced under inductive condition. There are so many vectors being used for this purpose, which ideally contain artificial ribosome binding site, transcription start site, transcription terminator, inducible promoter and a multiple cloning site (MCS) for cloning of desired gene at a particular site and a module for purification of the protein in the induced state. For the purification of protein of interest under inducible condition there are several criteria that can be used such as GST (Glutathion S transferase) fusion protein where protein of interest can be purified by Glutathion affinity column and further the protein can be obtained by the treatment of endopeptidase with GST peptide specificity. The other popular protein expression has 6×His tag which is coded by the sequence prior to the gene of interest, has affinity with Ni-affinity column and the protein of interest can be purified by imidazole elution. The pQE vectors (commercially available from Quigen) can be used for cloning the gene in three different frames such as 0, −2 and −1 frame(pQE-30, pQE-31 and pQE-32).

For this cloning, the AGT-SAL gene was first cloned in pBSKS(+) at EcoRI site (as a vector) whereas the gene was obtained from pTAdv-Sal and transformed to DH10B competent cells. The transformants were selected on LB Agar Amp(-IPTG/X-gal-) and white colonies were screened for the presence of insert using EcoRI and KpnI/SacI. The orientation of the insert was analysed using enzymes such as PstI, NcoI-SacI etc. The construct was named as pSV-SAL. From pSV-SAL, the gene was directionally cloned into pQE (pQE-30, pQE-31 and pQE-32) vectors by using AGT-SAL KpnI/SacI double digest and transformed in DH10B competent cells. The transformants were selected on LB Agar (Amp) and the transformants were screened. The recombinants were confirmed by digesting transformants plasmid with EcoRI and the three constructs were named as pExSV(1)SAL (have backbone of pQE-30), pExSV(2)SAL (have backbone of pQE-31) and pExSV(3)SAL (have backbone of pQE-32).

Further all constructs were transformed to M15 (commercially available from Quigen) competent cells for expression. M15 cells are specifically expression cells because of the presence of pREP4 which overproduces Lac repressor protein for Lac promoter and so the induction of gene of interest is tightly regulated.

The M15 cells with three constructs were grown till it reached to log phase, induction with IPTG was given and allowed for 3-4 hours. The cells were pelleted and dissolved in Tris-phosphate urea buffer(pH8.0). The samples of these were loaded to acrylamide gel with uninduced sample as control. After the protocol is standardized it will be deduced as which one of them is expressing the protein under induced conditions. The native AGT-SAL is purified. The protein was purified by Ni-NTA affinity column which has affinity for the 6×His tag and the elution was performed by buffer containing imidazole which has higher affinity for Ni matrix and then in turn compete with 6× His tagged protein and replaces them.

The structure and function of AGT-SAL-11 was predicted using computational Biology, (Bioinformatics). Bioinformatics is a theoretical approach where predictions are carried out using computer applications; the Biological Data generated from the Laboratories till date is the source for the Databases.

Although in most cases protein production is the ultimate output for the gene protein analysis techniques are currently less suitable for high throughput screening than Nucleic Acid analysis techniques. Thus RNA analysis are the most important at present.

To find any similar pattern or similar molecules in the database a program BLAST was performed but no significant results were obtained (using the gene sequence).

The subsequent tests mentioned below were performed to study the Protein level, the stage that actually determines the Function of a gene (AAF06789.1), having a sequence ID, SEQ ID 3.

The protein sequence was also subjected to similarity search initially with BLAST with BLOSUM-62, matrix but found no interesting results. BLOSUM stands for Block Summation matrix, which is used to find molecules, which are related to one another having similar sequences and accounts for similar functions as well.

For a more specific approach the tests were extended to FASTA, a stringent method for finding sequence similarity, in this attempt we could count on a group of hits which resembled AGT-SAL-11, using a matrix of BLOSUM-40. BLOSUM 40 is used to find distantly related molecules.

The Secondary structure of AGT-SAL-11 was predicted using the applications of Predict Protein server. The results obtained are as follows:
a) The molecule shows a mixture of αβ type of secondary structure.
b) There are sites for Glycosylation and Phosphorylation (mostly 0 Glycosylation with Serine or Threonine residues).
   Experimental Procedures
   I. Collection of Plant Materials.
a. Seeds of IR64 and Rasi, the two varieties of Indica rice chosen for the study were dehusked and good seeds were selected. These were surface sterilized using 70% ethyl alcohol for 1 minute and 2% sodium hypochlorite for 20 minutes. Surface sterilized seeds were washed repeatedly with sterile distilled water to remove traces of sterilizing agents.

Circular sterile filter papers were placed in autoclaved plastic petriplates and moistened with 20 ml sterile distilled water in the laminar flow hood. About 25 surface seeds were placed in each plate and the lid was covered and the plates were incubated at room temperature.

The seeds on an average took 2 days for germination. After germination the seedlings were allowed to grow for one week. The plates were constantly monitored for contamination. Since the plant material was to be used for RNA extraction, plates with any sign of contamination was discarded. Petriplates were irrigated whenever necessary.

Nine day old seedlings were used for inducing salt and dehydration stresses.
b. Induction of Salt Stress

For the induction of salt stress, the water in the petriplates containing 9 day old seedlings was replaced with 150 mM NaCI solution. One, two, four, eight and sixteen hours were collected by excising the endosperm and separating the seedling into root and shoot. The plant material was immediately frozen in liquid nitrogen and stored at −80 degrees Celsius for RNA isolation later on [IR64 S.S-IR64 rice variety under salt stress; RASI S.S-RASI rice variety under salt stress].
c. Induction of Moisture Stress

Moisture stress was induced by allowing nine days old seedlings to desiccate gradually in inflated plastic bags at room temperature. Loss of weight of the seedling was constantly monitored. Plastic bags were changed frequently to decrease humidity inside the bag. When the seedlings recorded 30% and 40% weight loss, samples were collected by excising the endosperm and separating the seedling into root and shoot and freezing them.
d. Controls

Unstressed nine day old seedlings of Rasi and IR 64, collected in the same manner as described above, were used as controls [IR64 C-IR64 rice variety kept as control; RASI C-RASI rice variety kept as control].

II. Isolation of Total RNA

a. Preparation of RNA extraction

The following precaution were taken to inhibit ribonucleases.
1. All glassware and heat resistant materials (pestle and mortar, forces etc. were baked overnight in an oven.
2. 0.1% DEPC (diethylpyrocarbonate) was added to all solutions (except those containing Tris) incubated overnight after thorough shaking and then autoclaved.
3. All plastic ware were treated with 10% hydrogen peroxide overnight, autoclaved and dried properly after use.
4. Clean disposable gloves were used at all stages of RNA extraction.
   b. RNA Isolation

Single step method of RNA isolation by acid Guanidium thiocyanate Phenol-chloroform extraction (Sacchi et. A1 1987) was employed to isolate total RNA. The procedure consisted of following steps:
1. 0.5 to 1 gm of tissue was ground in liquid nitrogen using pestle and mortar to make a fine powder.
2. To this 6 ml of freshly prepared extraction buffer was added and homogenized.
3. To the homogenate taken in a centrifuge tube, the following reagents were sequentially added and mixed thoroughly after addition of each reagent:
   a. ml of 2M sodium acetate (pH4)
   b. 10 ml of phenol (Saturated with DEPC treated water)
   c. 2 ml of chloroform: isoamyl alcohol (49:1) mixture

This was incubated on ice for 15 minutes and centrifuged at 8000 rpm for 12 minutes at 4 degrees C. The aqueous phase was carefully transferred to a fresh centrifuge tube and 10 ml of iso-propanol was added and mixed well and incubated at −20 degrees for 1 hour. The tube was centrifuged at 14,500 rpm for 20 minutes at 4° C. The pellet was re-suspended in 3 ml of extraction buffer and 3 ml of iso-propanol was added, mixed well and incubated at −20° C. for 1 hour. The tube was centrifuged at 14,500 rpm for 20 minutes at 4° C. The pellet was washed with 1 ml of 75% ethanol, centrifuged at 14,500 rpm for 15 minutes at 4° C. The pellet was dissolved in DEPC treated water and stored at −80° C.

a. Determination of RNA concentration

3 μl of RNA extract was taken in 1 ml of DEPC treated water for spectrophotometric quantification and purity analysis. Absorbance at 260 nm and 280 nm was taken using a “spectronic Genesis-5’ spectrophotometer. RNA concentrations were determined based on the relationship that an OD of 1 at 260 nm corresponds to 40 μg of RNA. RNA purity was assessed by calculating the A260/280 ratios (Table no. 1). The ratio should be close to 2 for a good RNA extraction.

b. Checking of RNA integrity by Submarine Agraose Gel electrophoresis.

A 100 ml 1.2% formaldehyde agarose gel was cast by melting 1.2 g of agarose (RNase free) in 73.3 ml of DEPC treated water. Just before pouring the gel, 10 ml of 10×MOPS/EDTA and 16.7 ml of formaldehyde (2.2M) was added.

30 μg of RNA was taken in 25 μl of the gel loading dye mixed well and heated at 65 degrees celsius for 15 minute on a dry bath and snap cooled on ice before loading on the gel.

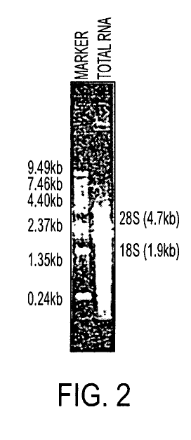

3 μl of 0.24 kb to 9.5 kb RNA ladder from GIBCO BRL containing a mixture of 6 synthetic poly (A) tailed RNAs (0.5 μg each) of sizes 9.49 kb, 7.46 kb, 4.40 kb, 2.37 kb, 1.35 kb and 0.24 kb was used as a marker for these gels (FIG. 2).

Horizontal or submarine agarose gel electrophoresis system was used. IX MOPS/EDTA was used as the electrode buffer. A potential difference of 5-10 volts per cm (distance between the electrodes) was used for the anionic run.

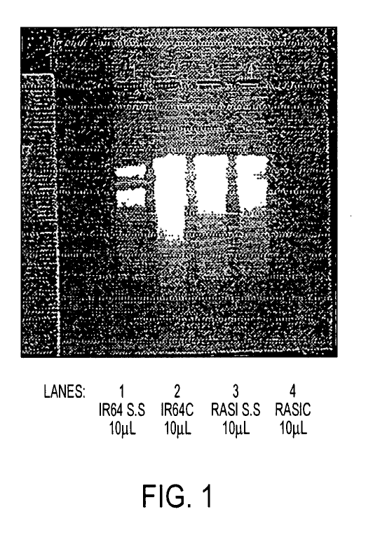

The two prominent RNA bands of sized 4.7 kb and 1.9 kb correspond to 28s and 18s ribosomal RNA activity (FIG. 1) Faint bands of 2.9 kn (23s chloroplast rRNA) and 1.5 kb (16s chloroplast rRNA) can also be visualized. 5s rRNA is about 120 bp and runs faintly below the dye front. The 240 bp RNA size marker comigrates with the Bromo-phenol blue dye front. The smear below the dye front also represents degraded RNA apart from tRNA and a small mRNA population. The rest of the RNA is the mRNA population. DNA(contamination) stays in the well hardly moves. A good RNA extract when runs on the gel shows minimum or no DNA in the well, distinct rRNA bands, prominent smear up to the dye front and a faint fizzy band below the dye front. (FIG. 1)

1. An isolated nucleic acid sequence comprising a polynucleotide having the sequence SEQ ID NO:1.

2. The nucleic acid sequence as claimed in claim 1 wherein said polynucleotide sequence encodes the polypeptide as shown in SEQ ID NO:2.

3. The nucleic acid sequence as claimed in claim 1 wherein said polynucleotide sequence is a full length AGTSAL 11 gene.

4. The nucleic acid sequence as claimed in claim 2 wherein said polypeptide sequence is a complete and mature AGTSAL 11 protein.

5. The nucleic acid sequence as claimed in claim 2 wherein said polypeptide has bi-functional units.

6. The nucleic acid sequence as claimed in claim 2 wherein said polypeptide has glycosylation and phosphorylation sites.

7. The nucleic acid sequence as claimed in claim 6 wherein said glycosylation is O glycosylation.

8. The nucleic acid sequence as claimed in claim 2 wherein said polynucleotide sequence has a mixture of αβ type of secondary structure.

9. The nucleic acid sequence as claimed in claim 2 wherein said polypeptide has similarity with proteinase inhibitors of Bowman Birk II type of super family of proteinase inhibitors.

10. A transgenic plant comprising a recombinant expression cassette comprising a plant promoter operably linked to the nucleic acid sequence as claimed in claim 1.

11. A method for conferring salt tolerance on a plant, the method comprising introducing into the plant a recombinant expression cassette comprising a plant promoter operably linked to the nucleic acid sequence as claimed in claim 1.


SEQUENCE LISTING (2 sequences)

SEQ ID NO: 1 - DNA, 673 bp, Rice
tttaccttgcctgctcggatggcagcaaactccatcttggggtgtggcgtgagcacaccaagaaattctcccctcagtggtttgcagctgtccatgccgctgttccattcattggaatgctgaggaaatctgtcaacatgcccaagactgccatggcattcaccatagcagcctccattgttggtcagacaatcgggtcgagggcggagcgcattcgtctgaaggcactggctgcaaagagcgacgctgattccaccaccgtggctgacatgtatccaaacaagactgcaaattgcagtgacaccgagggcaaggcatgggatccgctcgcgatgaagatgatggcgggacgggcttctggtggtgctgctgctccaacaccaagcatgtgtttctgattgctcactgattggaaaatttgtatctaccagtatccctggagagtggagagttgatattgagtctattttatcttgtgatgtaattgcctttgcttgtccctcagaagtattcgtttgtttgtgggatgagacaagtggaataagagtgctactatatacacgatcattctgttgttaagtttgccagttctgcagttcatgtatctgtaatttgatgatgctggatttctactatttatcaatcgtcattatactgtgtgtaaaaaaaaaaa

SEQ ID NO: 2 - protein, 143 aa, Rice
MVDTNFPISEQSETHAWCWSSSTTRSPSRHHLHRERIPCLALGVTAICSLVWIHVSHGGGISVALCSQCLQTNALRPRPDCLTNNGGCYGECHGSLGHVDRFPQHSNEWNSGMDSCKPLRGEFLGVLTPHPKMEFAAIRAGKV